(19) United States
(12) Patent Application Publication
(10) Pub. No.: US 2012/0238965 A1
(43) Pub. Date: Sep. 20, 2012

(54) MANUAL SWITCH FOR A CLOSED SUCTION TUBE

(76) Inventor: Ti-Li CHANG, Taichung City (TW)

(21) Appl. No.: 13/049,223

(22) Filed: Mar. 16, 2011

(51) Int. Cl. A61M 5/00

(52) U.S. Cl. 604/256

(57) ABSTRACT

A manual switch has a body, a button, a valve rod and a biasing member. The body has a valve mount having multiple holding ribs. The valve rod is mounted slidably in the valve mount, is pressed by the button and has a valve rod, a mounting tube and an O-ring. The rod body has an outer diameter smaller than the inner diameter of the valve mount. The mounting tube is mounted securely around the rod body and has an outer diameter being equal to the inner diameter of the valve mount and an outer surface abutting with the holding ribs in the valve mount. The O-ring is mounted around the rod body and abuts with the inner surface of the valve mount to provide a sealing effect to the valve rod and the valve mount.

### BACKGROUND OF THE INVENTION

1. Field of the Invention

The present invention relates to a switch, and more particularly to a manual switch for a closed suction tube for drawing secretion out from a human body and having an excellent sealing effect and a labor-saving capability.

2. Description of Related Art

A manual switch is mounted on a closed suction tube to manually control the suction tube to draw secretion out from a patient. With reference to FIG. 9, a conventional manual switch is connected to the suction tube and a vacuum pump/compressor and has a body 60, a button 62, a valve rod 64 and a spring 66. The body 60 has a connecting tube with two ends connected respectively with the suction tube and vacuum pump/compressor. The button 62 is mounted slidably on the body 60. The valve rod 64 is movable mounted in the body 60, is released upward by the button 62 and is mounted into the connecting tube to close the communication of the connecting tube. The spring 66 is mounted in the body 60 to provide a recoil force to the valve rod 64. When the button 62 is pressed, the valve rod 64 is moved downward to communicate the suction tube and the vacuum pump/compressor via the connecting tube. Consequently, the vacuum pump/compressor can provide a vacuum drawing force to draw secretion out from the patient through the suction tube.

However, the valve rod 64 of the conventional switch abuts with the inner surface in the body 60 with the whole outer surface of the valve rod 64 for closing the communication of the connecting tube, a large drag force caused by the viscosity of the air will occur when pressing the button 62. Therefore, the operation of the conventional switch is laborious. In addition, to keep air from leaking, the outer surface of the valve rod 64 has to contact with the inner surface in the body 60 tightly, but this will increase the drag force for pressing the button 62. Otherwise, a sealing effect cannot be achieved. Furthermore, although some conventional manual switch may further comprises a safety device to keep the button 62 from being pressed unintentionally, but the conventional safety device has a complicated structure and is trouble in operation.

To overcome the shortcomings, the present invention tends to provide a manual switch to mitigate or obviate the aforementioned problems.

### SUMMARY OF THE INVENTION

The main objective of the invention is to provide a manual switch for a closed suction tube being easily operated and providing an excellent sealing effect.

The manual switch has a body, a button, a valve rod and a biasing member. The body is hollow and has two ends, a connecting tube and a valve mount. The connecting tube is mounted through the body and has two ends protruding respectively from the ends of the body. The valve mount is hollow, is formed in the body, communicates with the connecting tube and has an inner diameter, an inner surface and multiple holding ribs formed around the inner surface, arranged in a circle and spaced from each other. The button is connected pivotally to the body at a pivotal position on the body. The valve rod is mounted slidably in the valve mount, is pressed by the button and has a valve rod, a mounting tube and an O-ring. The rod body has a top, a bottom and an outer diameter smaller than the inner diameter of the valve mount. The mounting tube is mounted securely around the bottom of the rod body and has an outer diameter being equal to the inner diameter of the valve mount to close the communication between the ends of the connecting tube and an outer surface abutting with the holding ribs in the valve mount. The O-ring is mounted around the rod body adjacent to the top of the rod body and abuts with the inner surface of the valve mount to provide a sealing effect to the valve rod and the valve mount. The biasing member is mounted in the valve mount and is connected to the valve rod to provide a recoil force to the valve rod.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a manual switch in accordance with the present invention connected with a closed suction tube;

FIG. 2 is a perspective view of the manual switch for a closed suction tube in FIG. 1;

FIG. 3 is an exploded perspective view of the manual switch in FIG. 2;

FIG. 4 is an cross sectional top view of the manual switch in FIG. 2;

FIG. 5 is a cross sectional side view of the manual switch in FIG. 2 in a locked condition;

FIG. 6 is an enlarged cross sectional end view of the manual switch in FIG. 2;

FIG. 7 is an operational perspective view of the manual switch in FIG. 2 in an unlocked condition;

FIG. 8 is an enlarged operational cross sectional end view of the manual switch in FIG. 1 when the button is pressed; and

FIG. 9 is a side view in partial section of a conventional manual switch in accordance with the prior art.

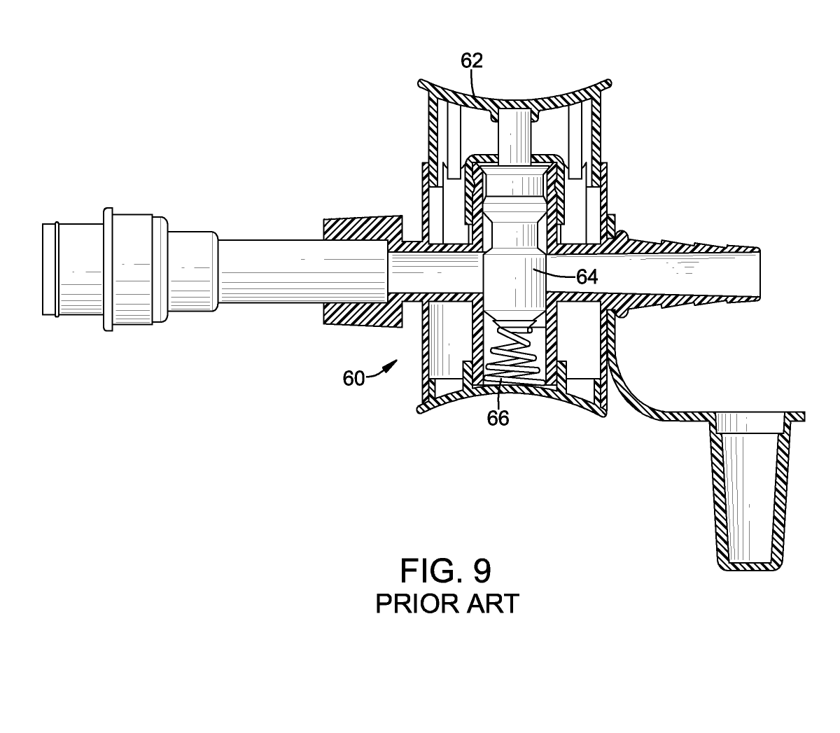

### DETAILED DESCRIPTION OF PREFERRED EMBODIMENT

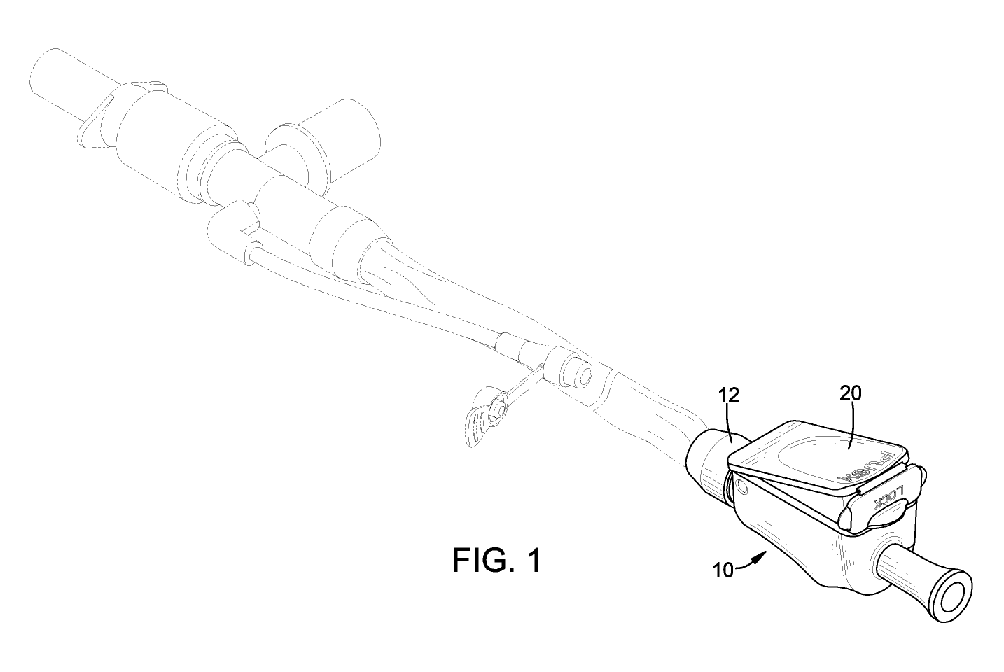

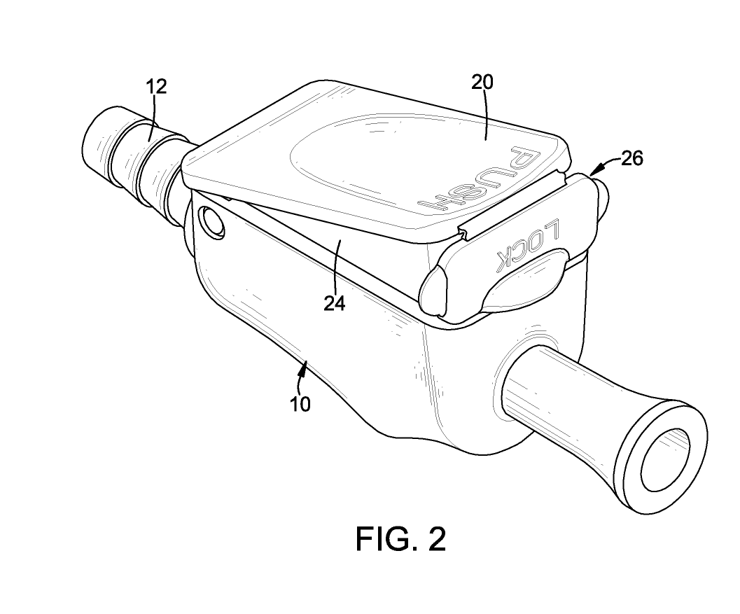

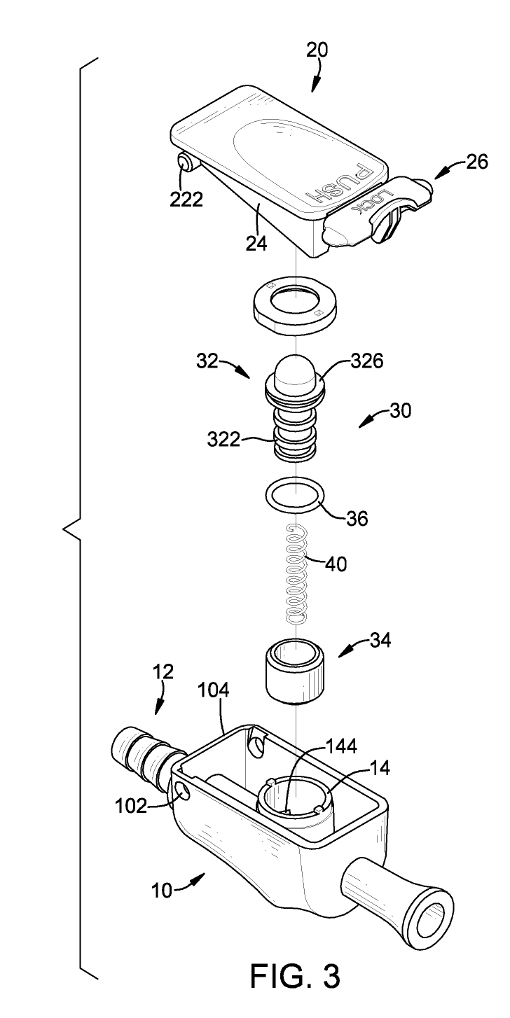

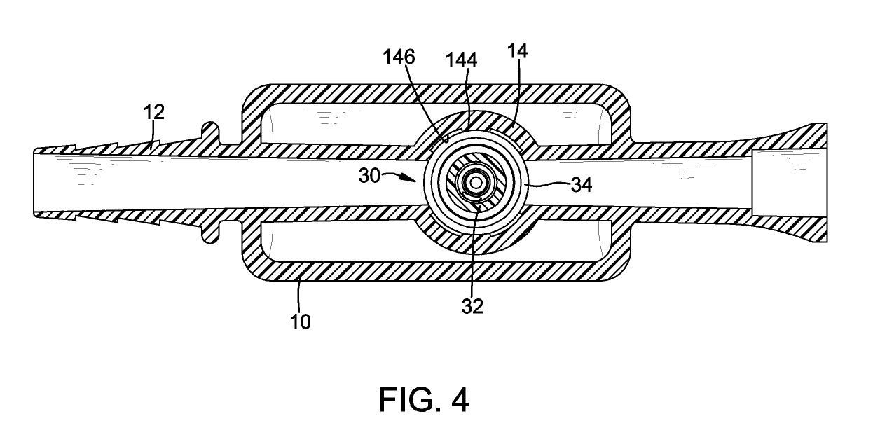

With reference to FIGS. 1 to 4, a manual switch in accordance with the present invention is connected to a closed suction tube and comprises a body 10, a button 20, a valve rod 30 and a biasing member 40. The body 10 is hollow and has two ends, a connecting tube 12 and a valve mount 14. The connecting tube 12 is mounted through the body 10 and has two ends protruding respectively from the ends of the body 10. The ends of the connecting tube 12 are connected respectively to a suction tube and a vacuum pump/compressor. The valve mount 14 is hollow, is formed in the body 10, communicates with the connecting tube 12 and has an inner diameter, an inner surface and multiple holding ribs 144. The holding ribs 144 are formed around the inner surface, are arranged in a circle and are spaced from each other to define multiple gaps 146 between the holding ribs 144.

The button 20 is connected pivotally to the body 10 at a pivotal position on the body 10. The pivotal position of the button 20 connected pivotally to the body 10 is at the top near one end of the body 10. The body 10 may further have an opening, an inner wall, two pivotal holes 102, an inclined edge 104 and a spring post 142. The opening is defined in the top of the body 10. The pivotal holes 102 are defined in or through the inner wall of the body 10. The inclined edge 104 is defined in the top of the body 10 at the end corresponding to the pivotal position. The spring post 142 is formed on and protrudes from the bottom of the valve mount 14.

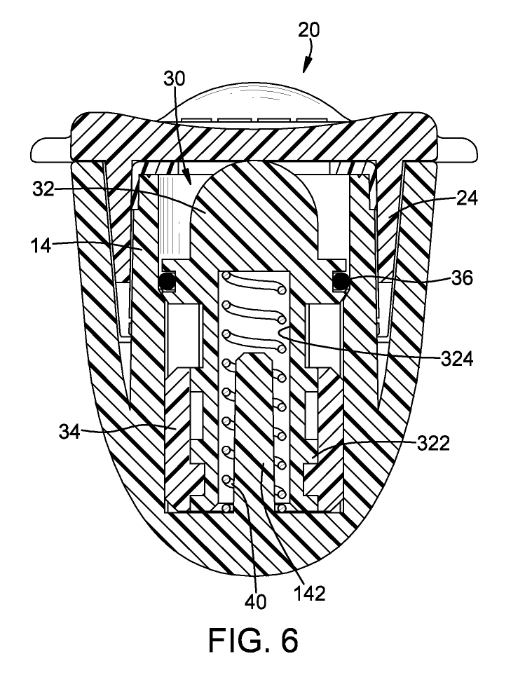

The button 20 has two ends, a bottom, a wall 24 and two pivotal arms 22. One end of the button 20 is connected pivotally to the body 10 and selectively abuts with the inclined edge 104 on the top of the body 10 to limit the pivotal angle of the button 20. With further reference to FIG. 6, the wall 24 is formed on and protrudes from the bottom of the button 20 and selectively abuts with the inner wall of the body 10 to provide a guiding effect to the button 20 to make the button 20 being pressed smoothly. The pivotal arms 22 are formed on and protrude from the bottom of the button 20 at the end corresponding to the pivotal position and correspond respectively to the pivotal holes 102 in the body 10. Each pivotal arm 22 has a pivotal boss 222 formed on the pivotal arm 22 and rotatably mounted in a corresponding one of the pivotal holes 102 in the body 10 to pivotally connect the button 20 to the body 10.

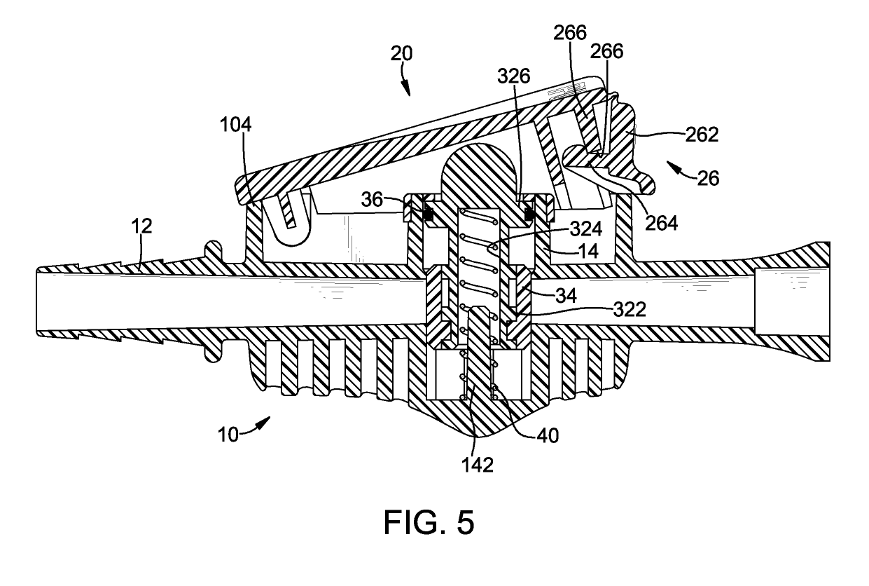

With further reference to FIG. 5, the valve rod 30 is mounted slidably in the valve mount 14, is pressed by the button 20 and comprises a rod body 32, a mounting tube 34 and an O-ring 36. The rod body 32 is mounted slidably in the valve mount 14 and has an outer diameter, a top, a bottom and a spring recess. The outer diameter of the rod body 32 is smaller than that of the valve mount 14. The spring recess 324 is defined in the bottom of the rod body 32. The mounting tube 34 is mounted securely around the bottom of the rod body 32 and an outer surface abutting with the holding ribs 144 in the valve mount 14. The mounting tube 34 has a length shorter than that of the rod body 32 to make the rod body exposed from the top of the mounting tube 34. To connect the mounting tube 34 securely with the rod body 32, the rod body 32 has multiple annular connecting ribs 322 formed around the rod body 32 near the bottom of the rod body 32. The mounting tube 34 has an outer diameter, an inner diameter larger than the outer diameter of the rod body 32 and an inner surface abutting securely with the connecting ribs 322 on the rod body 32. With the secure abutment between the inner surface of the mounting tube 34 and the connecting ribs 322, the mounting tube 34 is mounted securely with the rod body 32. In addition, the outer diameter of the mounting tube 34 is equal to the inner diameter of the valve mount 14 to close the communication between the ends of the connecting tube 12.

The O-ring 36 is mounted around the rod body 32 adjacent to the top of the rod body 32 and abuts with the inner surface of the valve mount 14 to provide an excellent sealing effect to the valve rod 32 and the valve mount 14. To mount the O-ring 36 on the rod body 32, the rod body 32 further has an annular supporting protrusion 326 formed around the rod body 32 near the top of the rod body 32 and the O-ring 36 is mounted around the annular supporting protrusion 326.

The biasing member 40 may be a spring, is mounted in the valve mount 14 and is connected to the valve rod 30 to provide a recoil force to the valve rod 30. The biasing member 40 has a bottom and a top. The bottom of the biasing member 40 abuts with the bottom of the valve mount 14 and is mounted around the spring post 142. The top of the biasing member 40 abuts with the rod body 32 of the valve rod 30 and is held in the spring recess 324 in the bottom of the rod body 32 of the valve rod 30.

In addition, the manual switch may further comprise a safety device 26 mounted on the button 20 at an end opposite to the pivotal position. The safety device 26 comprises an abutting tab 262 and a locking tab 266. The abutting tab 26 is pivotally connected to the end of the button 20 opposite to the pivotal position, selectively abuts against the top of the body 10 and has a hook 264 formed on the abutting tab 262. The locking tab 266 is formed on and protrudes from the bottom of the button 20 and has a locking hole 268. The locking hole 268 is defined in the locking tab 266 and selectively engages the hook 264 on the abutting tab 262 when the abutting tab 262 is pivoted to abut against the top of the body 10. With the abutment of the abutting tab 262 against the top of the body 10, the button 20 is kept from being pivoted relative to the body 10. Accordingly, the button 20 will not be pressed unintentionally, and the safety of using the suction tube is improved.

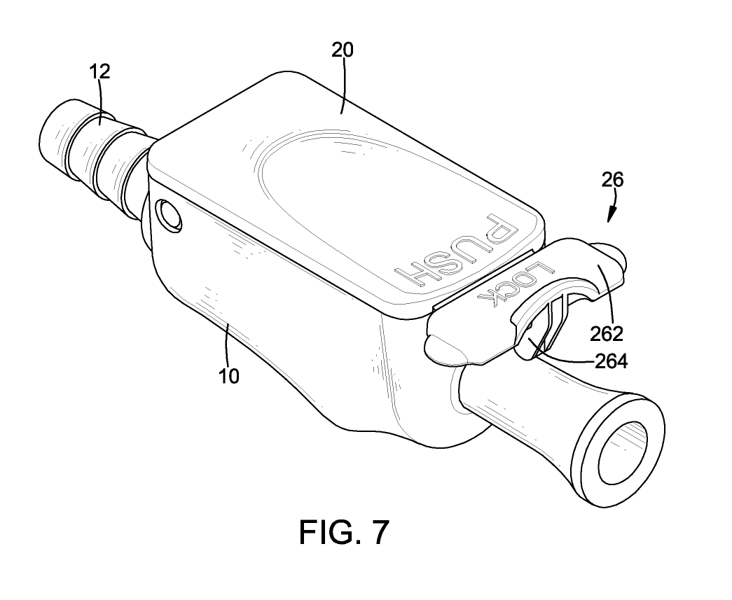

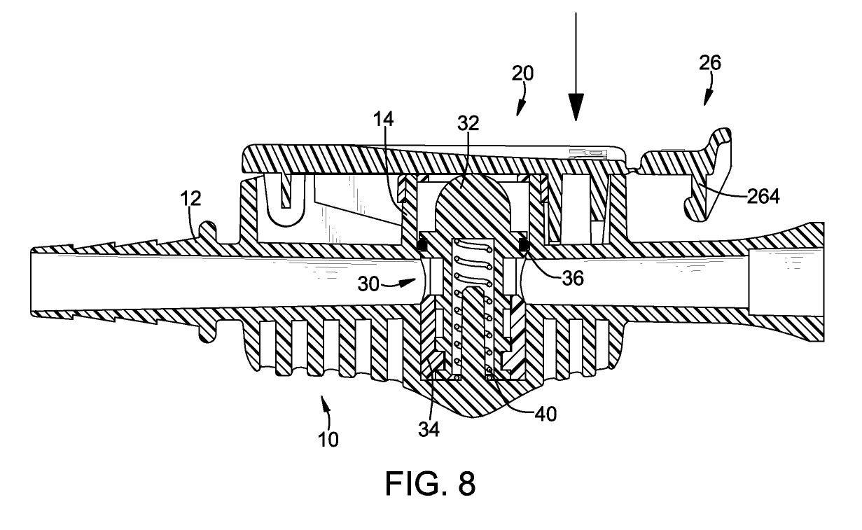

With further reference to FIGS. 7 and 8, in use, the abutting tap 262 is firstly pivoted to disengage the hook 264 from the locking hole 268 to enable the button 20 to be pivoted relative to the body 10. When the button 20 is pressed, the button 20 is pivoted relative to the body 10 and the valve rod 30 is moved downward along the valve mount 14 to make the rod body 32 exposed from the mounting tube 34 corresponding to the connecting tube 12. Because the outer diameter of the rod body 32 is smaller than the inner diameter of the valve mount 14, a passage is defined around the rod body 32 while the rod body 32 exposed from the mounting 14 corresponds to the connecting tube 12. Accordingly, the ends of the connecting tube 12 are communicated, such that the vacuum pump/compressor can provide a suction force to the suction tube to draw secretion out from a patient through the connecting tube 12 of the manual switch.

After the secretion-sucking process has been done, the button 20 is released, the valve rod 30 and the button 20 are moved to the original position with the recoil force provided by the biasing member 40 and the mounting tube 34 of the valve rod 30 closes the connecting tube 12 again. With the arrangement of the O-ring 36, an excellent sealing effect is provided, and air leakage can be efficiently prevented.

Because the outer surface of the mounting tube 34 abuts with the holding ribs 144 in the valve mount 14 but not abut with the whole inner surface of the valve mount 14, the contacting area between the mounting tube 34 and the valve mount 14 can be reduced. With the reduced contacting area, the drag force caused by the viscosity of the air can be efficiently reduced even completely eliminated due to the gaps 146 between the holding ribs 144, so a small force is needed to press the button 20 and the operation of the manual switch in accordance with the present invention is labor-saving and convenient.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A manual switch for a closed suction tube comprising:
a hollow body having
two ends;
a connecting tube mounted through the body and having two ends protruding respectively from the ends of the body; and
a hollow valve mount formed in the body, communicating with the connecting tube and having an inner diameter, an inner surface and multiple holding ribs formed around the inner surface, arranged in a circle and spaced from each other;
a button connected pivotally to the body at a pivotal position on the body;
a valve rod mounted slidably in the valve mount, pressed by the button and comprising
a rod body having a top, a bottom and an outer diameter smaller than the inner diameter of the valve mount;
a mounting tube mounted securely around the bottom of the rod body and having
an outer diameter being equal to the inner diameter of the valve mount to close the communication between the ends of the connecting tube; and
an outer surface abutting with the holding ribs in the valve mount;
an O-ring mounted around the rod body adjacent to the top of the rod body and abutting with the inner surface of the valve mount to provide a sealing effect to the valve rod and the valve mount;
a biasing member mounted in the valve mount and connected to the valve rod to provide a recoil force to the valve rod.

2. The manual switch as claimed in claim 1, wherein the body has an inner wall, a top and an opening defined in the top of the body;
the pivotal position of the button connected pivotally to the body is at the opening and near one of the ends of the body;
the button has
two ends;
a bottom; and
a wall formed on and protruding from the bottom of the button and selectively abutting with the inner wall of the body; and
the top of the valve rod protrudes out from the valve mount and abuts with the bottom at a position between the ends of the button.

3. The manual switch as claimed in claim 2, wherein
the body further has two pivotal holes defined in the inner wall of the body; and
the button further has two pivotal arms formed on and protruding from the bottom of the button at the end corresponding to the pivotal position and corresponding respectively to the pivotal holes in the body, and each pivotal arm has a pivotal boss formed on the pivotal arm and rotatably mounted in a corresponding one of the pivotal holes in the body.

4. The manual switch as claimed in claim 3 further comprising a safety device mounted on the button at the end opposite to the pivotal position and comprising
an abutting tab pivotally connected to the end of the button opposite to the pivotal position, selectively abutting against the top of the body and having a hook formed on the abutting tab;
a locking tab formed on and protruding from the bottom of the button and having a locking hole defined in the locking tab and selectively engaging the hook on the abutting tab when the abutting tab is pivoted to abut against the top of the body.

5. The manual switch as claimed in claim 4, wherein the biasing member has a bottom abutting with a bottom of the valve mount and a top abutting with the rod body of the valve rod.

6. The manual switch as claimed in claim 5, wherein
the valve mount has a spring post formed on and protruding from the bottom of the valve mount;
the rod body of the valve rod has a spring recess defined in a bottom of the rod body;
the bottom of the biasing member is mounted around the spring post; and
the top of the biasing member is held in the spring recess in the rod body of the valve rod.

7. The manual switch as claimed in claim 6, wherein the body further has an inclined edge defined in the top of the body at the end corresponding to the pivotal position and selectively abutting the end of the button corresponding to the pivotal position.

8. The manual switch as claimed in claim 7, wherein
the rod body has multiple annular connecting ribs formed around the rod body near the bottom of the rod body; and
the mounting tube has an inner surface abutting securely with the connecting ribs on the rod body to connect the mounting tube securely with the rod body.

9. The manual switch as claimed in claim 8, wherein the rod body of the valve rod further has an annular supporting protrusion formed around the rod body near the top of the rod body; and
the O-ring is mounted around the annular supporting protrusion.

10. The manual switch as claimed in claim 1, wherein
the body further has two pivotal holes defined in the inner wall of the body; and
the button further has two pivotal arms formed on and protruding from the bottom of the button at the end corresponding to the pivotal position and corresponding respectively to the pivotal holes in the body, and each pivotal arm has a pivotal boss formed on the pivotal arm and rotatably mounted in a corresponding one of the pivotal holes in the body.

11. The manual switch as claimed in claim 1 further comprising a safety device mounted on the button at the end opposite to the pivotal position and comprising
an abutting tab pivotally connected to the end of the button opposite to the pivotal position, selectively abutting against the top of the body and having a hook formed on the abutting tab;
a locking tab formed on and protruding from the bottom of the button and having a locking hole defined in the locking tab and selectively engaging the hook on the abutting tab when the abutting tab is pivoted to abut against the top of the body.

12. The manual switch as claimed in claim 1, wherein the biasing member has a bottom abutting with a bottom of the valve mount and a top abutting with the rod body of the valve rod.

13. The manual switch as claimed in claim 12, wherein
the valve mount has a spring post formed on and protruding from the bottom of the valve mount;
the rod body of the valve rod has a spring recess defined in a bottom of the rod body;
the bottom of the biasing member is mounted around the spring post; and
the top of the biasing member is held in the spring recess in the rod body of the valve rod.

14. The manual switch as claimed in claim 1, wherein the body further has an inclined edge defined in the top of the body at the end corresponding to the pivotal position and selectively abutting the end of the button corresponding to the pivotal position.

15. The manual switch as claimed in claim 1, wherein
the rod body has multiple annular connecting ribs formed around the rod body near the bottom of the rod body; and
the mounting tube has an inner surface abutting securely with the connecting ribs on the rod body to connect the mounting tube securely with the rod body.

16. The manual switch as claimed in claim 15, wherein the rod body of the valve rod further has an annular supporting protrusion formed around the rod body near the top of the rod body; and
the O-ring is mounted around the annular supporting protrusion.

17. The manual switch as claimed in claim 1, wherein the rod body of the valve rod further has an annular supporting protrusion formed around the rod body near the top of the rod body; and
the O-ring is mounted around the annular supporting protrusion.
